(19) United States
(12) Patent Application Publication
(10) Pub. No.: US 2005/0197356 A1
(43) Pub. Date: Sep. 8, 2005

(54) Non-immunosuppressive immunophilin ligands as neuroprotective and/or neuroregenerative agents

(75) Inventors: Edmund Idris Graziani, Ridgewood, NJ (US); Kevin Pong, Robbinsville, NJ (US)

Correspondence Address: HOWSON AND HOWSON;CATHY A. KODROFF, ONE SPRING HOUSE CORPORATE CENTER, BOX 457, SPRING HOUSE, PA, 19477, US

(73) Assignee: Wyeth, Madison, NJ (US)

(21) Appl. No.: 11/066,031

(22) Filed: Feb. 25, 2005

Related U.S. Application Data
(60) Provisional application No. 60/549,430, filed on Mar. 2, 2004.

(51) Int. Cl. A61K 31/4745

(52) U.S. Cl. 514/291; 540/456

(57) ABSTRACT

This invention relates, in part, to the use of non-immunosuppressive immunophilin ligands, such as meridamycin, in treating neurological disorders.

### CROSS-REFERENCE TO RELATED APPLICATION

This is a non-provisional application claiming the benefit under 35 USC 119(e) of U.S. Provisional Patent Application No. 60/549,430, filed Mar. 2, 2004.

### BACKGROUND OF THE INVENTION

The present invention relates generally to neuroprotective and/or neuroregenerative agents.

Immunophilins are proteins found in the immune systems and nervous systems of, e.g., bacteria, yeast, and various mammalian cells. Classes of immunophilins include cyclophilins and FK506-binding proteins (e.g., FKBPs). Cyclosporin A is a macrolide immunphilin ligand that binds to cyclophilins. Other macrolide immunophilin ligands, such as meridamycin, FK506, and rapamycin, are understood to bind to FKBPs.

One way to describe intracellular functions of immunophilins is by identification of their enzymatic activity. Functions of FKBPs, for example, can be described in terms of their rotamase (i.e., petidy-prolyl cis-trans isomerase) activity.

FK506 and rapamycin are immunosuppressive immunophilin ligands. Meridamycin, on the other hand, is non-immunosuppressive. Salituro, et al., Tetrahedron Letters, Vol. 36, No. 7, 997-1000 (1995). In fact, meridamycin is an antagonist of both FK506 and rapamycin. (WO 94/18207).

Other non-immunosuppressive immunophilins are described by Steiner et al. (U.S. Pat. No. 6,500,843) who discuss using neurotrophic pipecolic acid derivative compounds having an affinity for FKBP-type immunophilins as inhibitors of the enzyme activity associated with immunophilin proteins, and particularly inhibitors of peptidyl-prolyl isomerase or rotamase enzyme activity to stimulate or promote neuronal growth or regeneration.

Meridamycin has been identified for uses such as an antidote for an overdose of macrophilin-binding-immunosuppressants such as FK506 or rapamycin, a steroid potentiator, and/or an anti-infective agent for infections or infectious diseases caused by organisms producing MIP (macrophage infectivity potentiator) or MIP-like factors. (WO 94/18207). In addition, meridamycin may be useful in the treatment of inflammatory/hyperproliferative skin diseases. (WO 94/18207).

It is desirable to find agents that are neurotrophic, e.g., neuroprotective and/or neuroregenerative. A need exists in the art, therefore, to provide compounds, and therapeutic drugs comprising such compounds, for treatment of, for example, neurological disorders, that are non-immunosuppressive and have relatively low toxicity.

### SUMMARY OF THE INVENTION

In one aspect, this invention relates to the use of immunophilin ligands, particularly non-immunosuppressive ligands such as a meridamycin, or a pharmaceutically acceptable salt thereof, in treating neurological disorders. The invention is useful in preparation of compositions, including medicaments, further comprising one or more pharmaceutically acceptable carriers, excipients, or diluents, containing a meridamycin or pharmaceutically acceptable salts thereof.

In one aspect, the present invention provides methods for treatment of neurological disorder comprising administering to a mammal an effective amount of a meridamycin compound. Such methods of treatment can further include identifying mammals suffering from the neurological disorder. In certain embodiments, the methods of the invention include assessing a degree of neurodegeneration in a mammal, before administering the compound of a meridamycin, after administering it, or both before and after administering it.

Although not intending to be limited with respect to the nature of treatments embraced by the present invention, it is preferred to use a meridamycin compound for treatment of conditions of the central nervous system. Conditions affecting the central nervous system include, but are not limited to, epilepsy, stroke, cerebral ischemia, cerebral palsy, Alper's disease, Parkinson's disease, Alzheimer's disease, Huntington's disease, amyotrophic lateral sclerosis (ALS), multiple sclerosis, dementia with Lewy bodies, Rhett syndrome, neuropathic pain, spinal cord trauma, or traumatic brain injury.

In preparing medicaments of the invention, a meridamycin compound or salts thereof can be admixed with one or more pharmaceutically acceptable carriers, excipients, or diluents. Pharmaceutically acceptable formulations comprising compounds of the present invention can be of any suitable delivery vehicles, including but not limited to solid dosage forms, liquid dosage forms, aerosols, and the like.

Still other aspects and advantages of the invention will be readily apparent from the following detailed description of the invention.

### BRIEF DESCRIPTION OF THE DRAWINGS

FIG. 1 is a proton NMR spectrum of the compound of formula (I) in CH₃OD at 400 MHz.

### DETAILED DESCRIPTION OF THE INVENTION

The present invention provides the use of meridamycin and pharmaceutically acceptable salts thereof functions as a neurotrophic agent, i.e., a compound displaying neuroprotective and/or neuroregenerative activities.

As used herein, the term “a meridamycin” refers to compounds having the core structure of formula (I)

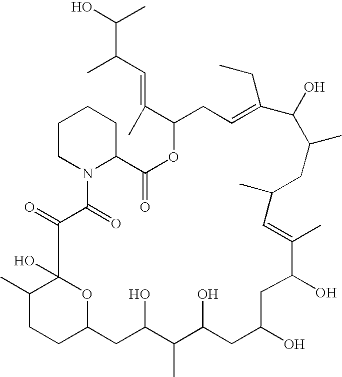

and pharmaceutically acceptable salts thereof.

The terms “pharmaceutically acceptable salts” and “pharmaceutically acceptable salt” refer to salts derived from organic and inorganic acids such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, and similarly known acceptable acids.

For purposes of convenience, reference is made throughout the specification to compounds of formula (I). However, it will be understood that a salt thereof, or a meridamycin as defined herein can be produced or used as described with reference to formula (I). According to this invention, the term “meridamycin” or “a meridamycin compound” further encompasses a compound having the following physicochemical characteristics:

Apparent Molecular Formula: C₄₅H₇₅O₁₂N

Molecular Weight: Positive Ion Electrospray m/z=844.8 (M+Na)⁺; Negative Ion Electrospray MS m/z=821.1 (M−H)⁻; High Resolution Fourier Transform MS m/z=822.53637 (M+H)⁺

Ultraviolet Absorption Spectrum: λₘₐₓ nm (acetonitrile/water)=210 nm, end absorption.

Optical Rotation [α]²⁵_{D}−1.4 (c 1.0, MeOH)

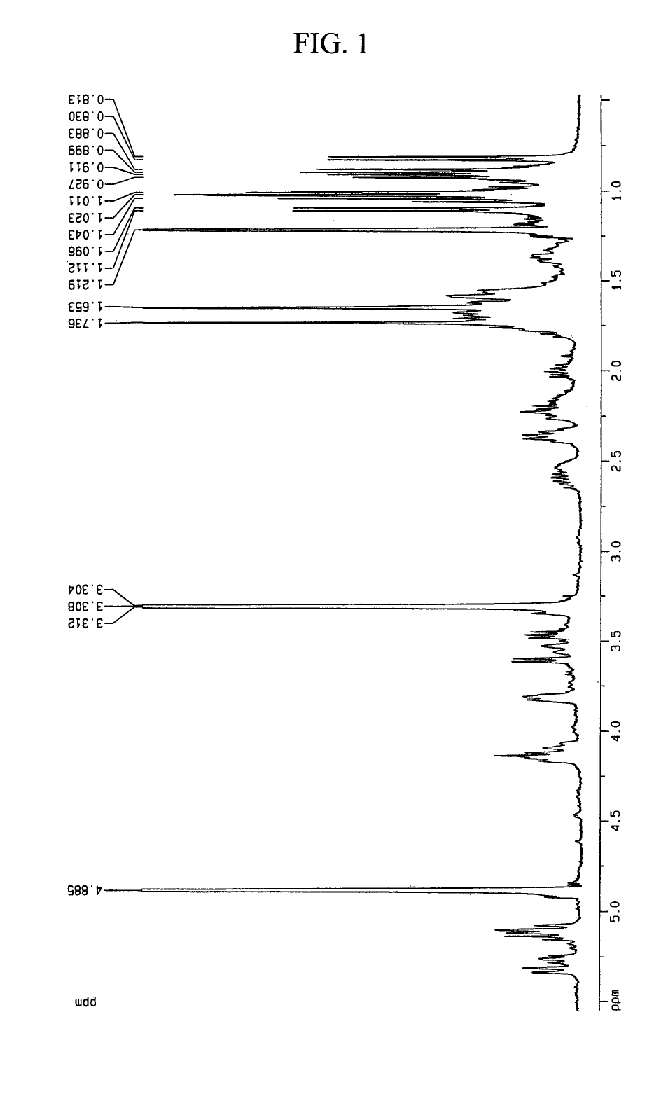

Proton Magnetic Resonance Spectrum: (400 mHz CH₃OD): See FIG. 1.

While shown without respect to stereochemistry in formula (I), the compounds of formula (I) can contain one or more chiral centers. Reference to “compound of formula (I)” is understood to include any compound of the implicated structural formula including all stereoisomers thereof.

In one embodiment, methods of producing the compounds utilize isolates (cells) of actinomycete strains that are classified in the genus Streptomyces by 16S rDNA sequence comparison. The isolates of the actinomycete strains produce no aerial mycelium and tan mycelium with no soluble pigment when grown on agar medium, e.g., ATCC agar medium as described herein, No. 172 or 174 (ATCC Media Handbook, 1^{st} edition, 1984). Alternatively, other suitable media may be purchased commercially, e.g., Sigma (St. Louis, Mo.). In a further embodiment, the isolates of the actinomycete strains produce at least one compound which is a compound of Formula I. In a further embodiment the isolates of the actinomycete strains produce both a compound of Formula I.

A compound of formula (I) preferably is obtained through purification of fermentation broths of the soil actinomycete strain LL-C31037 (NRRL 30721) or BD240. Reference to “compound of formula (I)” is understood to include any compound of the structure of (I) including all isomers thereof. Meridamycin, for example, was purified from an extract of fermentations of strain LL-C31037 or BD240, as described herein.

For the production of the neuroprotective compound (I) the present invention is not limited to a particular organism, for example, Streptomyces species designated LL-C31037 and BD240. In fact, it is desired and intended to include the use of naturally-occurring mutants of this organism, as well as induced mutants produced from this organism by various mutagenic means known to those skilled in the art, for example, exposure to nitrogen mustard, X-ray radiation, ultraviolet radiation, N′-methyl-N′-nitro-N-nitrosoguanidine, or actinophages. It is also desired and intended to include inter- and intraspecific genetic recombinants produced by genetic techniques known to those skilled in the art such as, for example, conjugation, transduction and genetic engineering techniques.

The methods of preparing the compound of formula (I) preferably involve growth in fermentation of the actinomycete strains LL-C31037 and BD240.

In one embodiment, the invention utilizes actinomycete strain LL-C31037, which was deposited pursuant to the provisions of the Budapest Treaty with the Agricultural Research Service Culture Collection (NRRL), 1815 North University Avenue, Peoria, Ill. 61604, on Mar. 1, 2004 and assigned the NRRL designation number 30721. In another embodiment, the invention utilizes actinomycete strain BD240, which was deposited pursuant to the provisions of the Budapest Treaty with the Agricultural Research Service Culture Collection (NRRL), 1815 North University Avenue, Peoria, Ill. 61604, on Jan. 19, 2005, and assigned the NRRL designation number 30810. The invention may further utilize isolates of the novel strains of the invention and derivatives, mutants, recombinants, and modified forms thereof which are characterized by the ability to produce a compound of formula (I). In one embodiment, the derivatives, mutants, recombinants, and modified forms thereof are further characterized by one or more of the following characteristics: not producing aerial mycelium, producing substrate mycelium which is tan and producing no soluble pigment.

Additional disclosure relating to, for example, actinomycete strain LL-C31037 is provided in commonly assigned U.S. Provisional Patent Application 60/549,480, filed Mar. 2, 2004, entitled “Macrolides And Compositions And Methods For Producing Same” bearing attorney docket AM101593. Additional disclosure relating to this, and actinomycete strain BD240 are provided in the corresponding US non-provisional, bearing the same title and docket number, and filed simultaneously herewith, the disclosure of which is incorporated herein by reference.

Fermentation conditions to culture Streptomyces species LL-C31037 and BD240 for production of macrolide compounds including compound (I) can be performed in flasks. Alternatively, production of higher volumes can be performed in fermentors under similar conditions.

Media useful for the cultivation of Streptomyces sp. LL-C31037 and BD240 and the production of the macrolide compounds include assimilable carbon sources such as, for example, dextrose, sucrose, glycerol, molasses, starch galactose, fructose, corn starch, malt extract and combinations thereof; an assimilable source of nitrogen such as, for example, ammonium chloride, ammonium sulfate, ammonium nitrate, sodium nitrate, amino acids, protein hydrolysates, corn steep liquor, casamino acid, yeast extract, peptone, tryptone and combinations thereof; and inorganic anions and cations such as, for example, potassium, sodium, sulfate, calcium, magnesium, chloride. Trace elements such as, for example, zinc, cobalt, iron, boron, molybdenum, and copper are supplied as impurities of other constituents of the media. Aeration in tanks and bottles is supplied by forcing sterile air through or onto the surface of the fermenting medium. A mechanical impeller provides further agitation in tanks. An antifoam agent such as polypropylene glycol can be added as needed.

Fermentation conditions for cultivation under controlled conditions of an actinomycete strain such as described herein, to produce a neuroprotective and neuroregenerative compound of formula (I) in growth media.

In one embodiment, a fermentation production medium is prepared by combining dextrose in a weight percentage of about 1% to about 2%; about 1% to about 3% of a soy source, about 0.25% to about 1% of yeast, about 0.1% of a calcium source, about 5% to about 10%, and preferably 6% to 8% maltodextrin, and, optionally, proline, from 0 to 0.5%. Optionally, other components may be included. Suitably, the media is adjusted to a pH in the range of about 6.5 to 7.5, and preferably about 6.8 to 7. Typically, the culture is allowed to ferment with suitable agitation and aeration. Alternatively, other suitable fermentation media may be prepared by one of skill in the art substituting other appropriate carbon source or other components and/or purchased commercially. See, generally, e.g., Sigma Aldrich (St. Louis, Mo.); G. J. Tortora e tal, Microbiology: An Introduction Media Update (Benjamin Cummings Publishing Co; Oct. 1, 2001); Maintaining Cultures for Biotechnology and Industry, eds. J. C. Hunter-Cevera and A. Bet (Academic Press, Jan 25, 1996).

After about 5 to 10 days, and preferably about 7 days of fermentation, the cells from the culture are pelleted by centrifugation. In one embodiment, the cells are extracted with a suitable solvent, e.g., ethyl acetate. The extract is concentrated in vacuo and resuspended in a minimum volume of a suitable solvent, e.g., methanol. The solution is loaded onto a reverse phase silica column and eluted with 20%-100% methanol in water. The fractions eluting from 60% methanol to 100% methanol are concentrated in vacuo. The prolylmeridamycin containing fractions are separated by suitable means, e.g., chromatographic methods.

In another embodiment, the supernatant is mixed with a suitable resin and allowed to rest from about 8 to 16 hours. Thereafter, the resin is washed with a suitable solvent, e.g., methanol, and the filtrate collected. To the cell pellet, an ethyl acetate—methanol mixture is added. This is repeatedly shaken and centrifuged, and the supernatant collected. The cell supernatant and the broth methanol filtrate are combined and concentrated in vacuo. Crude extract is adsorbed onto silica, and fractionated by vacuum liquid chromatography (VLC). The compound is eluted with a suitable solvent, e.g., methanol in dichloromethane. This extract is concentrated, adsorbed onto silica and loaded onto a flash silica column. The compound is eluted with a suitable solvent, concentrated and further purified by column chromatography.

The presence of the compound of formula I in the crude or semi-purified material can be confirmed by conventional methods, e.g., liquid chromatography mass spectrometric (LCMS) analysis of fractions. These fractions may be pooled and further purified by chromatographic methods, and optionally concentrated, e.g., in vacuo.

The resulting purified compounds are free of cells and cellular materials, by-products, reagents, and other foreign material as necessary to permit handling and formulating of the compound for laboratory and/or clinical purposes. It is preferable that purity of the compounds used in the present invention have a purity of greater than 80% by weight; more preferably at least 90% by weight, even more preferably greater than 95% by weight; yet even more preferably at least 99% by weight. In one embodiment, the invention provides compositions containing the compounds useful in the invention, regardless of how such compounds are produced.

The terms “effective amount” and “therapeutically effective amount,” as used herein, refer to the amount of a compound of formula (I) that, when administered to a patient, is effective to at least partially ameliorate a condition from which the patient is suspected to suffer. Although not intending to be limited in its therapeutic applications, it is desirable to use a compound of formula (I) for treatment of conditions of the central nervous system. Conditions affecting the central nervous system include, but are not limited to, epilepsy, stroke, cerebral ischemia, cerebral palsy, Alper's disease, Parkinson's disease, Alzheimer's disease, Huntington's disease, amyotrophic lateral sclerosis (ALS), dementia with Lewy bodies, Rhett syndrome, neuropathic pain, spinal cord trauma, or traumatic brain injury.

The term “subject” or “patient,” as used herein, refers to a mammal, which may be a human or a non-human animal.

The terms “administer,” “administering,” or “administration,” as used herein, refer to either directly administering a compound or composition to a patient, or administering a prodrug derivative or analog of the compound to the patient, which will form an equivalent amount of the active compound or substance within the patient's body.

Neurological disorders according to the invention include, but are not limited to, various peripheral neuropathic and neurological disorders related to neurodegeneration including, but not limited to: trigeminal neuralgia, glossopharyngeal neuralgia, Bell's palsy, myasthenia gravis, muscular dystrophy, amyotrophic lateral sclerosis (ALS), multiple sclerosis, progressive muscular atrophy, progressive bulbar inherited muscular atrophy, herniated, ruptured or prolapsed vertebral disk syndromes, cervical spondylosis, plexus disorders, thoracic outlet destruction syndromes, peripheral neuropathies such as those caused by lead, acrylamides, gamma-diketones (glue-sniffer's neuropathy), carbon disulfide, dapsone, ticks, porphyria, Gullain-Barre syndrome, dimentia, Alzheimer's disease, Parkinson's disease, and Huntington's chorea.

It is desirable to use meridamycin in treatments of disorders of the central nervous system. Conditions affecting the central nervous system include, but are not limited to, epilepsy, stroke, cerebral ischemia, cerebral palsy, Alper's disease, Parkinson's disease, Alzheimer's disease, Huntington's disease, amyotrophic lateral sclerosis (ALS), dementia with Lewy bodies, Rhett syndrome, neuropathic pain, spinal cord trauma, or traumatic brain injury.

Specific situations in which neurotrophic therapy is indicated to be warranted are with central nervous system disorders, Alzheimer's disease, Parkinson's disease, Huntington's disease, multiple sclerosis, amyotrophic lateral sclerosis, traumatic brain injury, spinal cord injury, epilepsy, aging, inflammatory disorders, rheumatoid arthritis, autoimmune diseases, respiratory distress, emphysema, psoriasis, adult respiratory distress syndrome, central nervous system trauma, and stroke.

The compounds of this invention are also useful in providing cognition enhancement, and in treating or inhibiting senile dementias, dementia with lewy bodies, mild cognitive impairment, Alzheimer's disease, cognitive decline, neurodegenerative disorders, providing neuroprotection or cognition enhancement.

When administered for the treatment or inhibition of a particular disease state or disorder, it is understood that the effective dosage may vary depending upon the particular compound utilized, the mode of administration, the condition, and severity thereof, of the condition being treated, as well as the various physical factors related to the individual being treated. Effective administration of the compounds of this invention may be given at monthly, weekly, or daily, or other suitable intervals. For example, a parenteral dose may be delivered on a weekly basis at a dose of about 10 mg to about 1000 mg, about 50 mg to about 500 mg, or about 100 mg to about 250 mg per week. A suitable oral dose may be greater than about 0.1 mg/day. Preferably, administration will be greater than about 10 mg/day, more specifically greater than about 50 mg/day in a single dose or in two or more divided doses. The oral dose generally will not exceed about 1,000 mg/day and more specifically will not exceed about 600 mg/day. The projected daily dosages are expected to vary with route of administration.

Such doses may be administered in any manner useful in directing the active compounds herein to the recipient's bloodstream, including orally, via implants, parenterally (including intravenous, intraperitoneal and subcutaneous injections), rectally, intranasally, vaginally, and transdermally.

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the compounds directly to the airways in the form of an aerosol.

The compounds of this invention may also be administered parenterally or intraperitoneally. Solutions or suspensions of these active compounds as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparation contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

The invention further provides products, including packaging, containing the compounds formulated for delivery. In another aspect, the invention provides kits including, e.g., needles, syringes, and other packaging, for delivery of the compound of the invention. Optionally, such a kit may include directions for administration of the drug, diluent, and or a carrier for mixing of a solid form of a compound of the invention.

The reagents used in the preparation of the compounds of this invention can be either commercially obtained or can be prepared by standard procedures described in the literature.

The preparation of representative examples of this invention are described in the following examples.

### EXAMPLE 1

### Fermentation Conditions for Actinomycete Strain LL-C31037

Fermentation conditions for cultivation under controlled conditions of an actinomycete strain designated LL-C31037 produce a neuroprotective and neuroregenerative compound of formula (I) in the growth media.

The actinomycete strain is maintained in the culture collection of Wyeth Research, Pearl River, N.Y. 10965, as culture LL-C31037. A viable culture of this microorganism has been deposited under the Budapest Treaty with the Patent Culture Collection, Northern Regional Research Laboratory (NRRL), U.S. Department of Agriculture, Peoria, Ill. 61604, and added to its permanent collection. Culture LL-C31037 has been assigned the NRRL accession number 30721, deposited on Mar. 1, 2004.

Culture of actinomycete strain LL-C31037 on agar plates, e.g., ATCC agar medium No. 172, produces no aerial mycelium. The substrate mycelium is tan and no soluble pigment is produced. The 16S rDNA sequence was determined for strain LL-C31037 following isolation and direct sequencing of the amplified gene. The nucleotide sequence was aligned with the sequences of previously studied streptomycetes, and phylogenetic trees were generated by using two neighbor-joining tree algorithms. The 16S rDNA sequence supported classification of the strain in the genus Streptomyces.

Fermentation conditions to culture Streptomyces species LL-C31037 for production of compound (I) were performed in flasks. Alternatively, production of higher volumes was performed in fermentors under similar conditions.

A. Flask Fermentation

A seed medium of the following formulation was be prepared by combining: dextrose (added after autoclaving), 1%; soluble starch, 2%; yeast extract, 0.5%; N-Z amine type A (Sheffield), 0.5%; calcium carbonate, 0.1%; pH at 7.0.

Ten ml of seed medium in a 25×150 mm glass tube was inoculated with two loopfuls of cell mass of LL-C31037 cultured on ATCC agar medium #172. Sufficient inoculum from the agar culture was used to provide a turbid seed after 72 hours of growth. The primary seed tube was incubated for 72 hours at 28° C., at 200 rpm using a gyro-rotary shaker with a 2-inch orbit. The primary seed (7 ml) was then used to inoculate a 250 ml Erlenmeyer flask containing 30 ml of seed medium. This secondary seed flask was incubated for 24 hours at 28° C., 200 rpm using a gyro-rotary shaker (2-inch orbit).

A fermentation production medium of the following formulation was prepared by combining: dextrose (added after autoclaving), 1%; maltrin M180, 6%; soyflour, 1%; yeast extract, 0.6%; Gamaco (CaCO₃), 0.1%; pH at 7.0.

One ml of secondary seed culture was inoculated into 50 ml of fermentation production medium in 250 ml Erlenmeyer flasks. These production flasks were incubated for 7 days at 26° C., 200 rpm using a gyro-rotary shaker (2-inch orbit).

B. Fermentor Fermentation

A seed medium of the following formulation was prepared by combining: dextrose (added after autoclaving), 2%; soluble starch, 2%; yeast extract (Difco), 0.3%; wheat hydrolysate WGE80M(DMV International), 0.5%; soy hydrolysate SE50MAF (DMV International), 1.5%; pH at 6.8 to 7.0.

One ml of frozen seed culture was inoculated into 1 liter of seed medium in a 4 liter Erlenmeyer flask. This seed flask was incubated for 72 hours at 30° C. at 250 rpm using a gyro-rotary shaker (2-inch orbit).

A fermentation production medium of the following formulation was prepared by combining: dextrose (added after autoclaving), 2%; maltrin M500, 8%; nutrisoy (GPC), 1%; yeast extract (Difco), 0.6%; Gamaco (CaCO₃), 0.1%; Macol P2000, 0.2%; pH at 6.8 to 7.0.

The 1 liter of seed culture was inoculated into 60 liters of fermentation production medium in a 70 liter fermentor. The fermentation was incubated for 5 days at 26° C., agitation at 350-550 rpm, aeration at 0.5-0.75 volvol⁻¹min⁻¹(VVM).

### EXAMPLE 2

### Fermentation Conditions for Actinomycete Strain BD240

Fermentation conditions for cultivation under controlled conditions of an actinomycete strain designated BD240 produce a neuroprotective and neuroregenerative compound of formula (I) in the growth media.

The actinomycete strain is maintained in the culture collection of Wyeth Research, Pearl River, N.Y. 10965, as culture BD240. A viable culture of this microorganism has been deposited under the Budapest Treaty with the Patent Culture Collection, Northern Regional Research Laboratory (NRRL), U.S. Department of Agriculture, Peoria, Ill. 61604, and added to its permanent collection. Culture BD240 has been assigned the NRRL accession number 30810, deposited on Jan. 19, 2005.

Culture of actinomycete strain BD240 on agar plates, e.g., ATCC agar medium No. 174, produces no aerial mycelium. The substrate mycelium is tan and no soluble pigment is produced. The 16S rDNA sequence was determined for strain BD240 following isolation and direct sequencing of the amplified gene. The nucleotide sequence was aligned with the sequences of previously studied streptomycetes, and phylogenetic trees were generated by using two neighbor-joining tree algorithms. The 16S rDNA sequence supported classification of the strain in the genus Streptomyces.

Fermentation conditions to culture Streptomyces species BD240 for production of compound (I) were performed in flasks. Alternatively, production of higher volumes was performed in fermentors under similar conditions.

A. Flask Fermentation

A seed medium of the following formulation was prepared by combining: dextrose (added after autoclaving), 1%; soluble starch, 2%; yeast extract (Difco), 0.3%; wheat hydrolysate WGE80M(DMV International), 0.5%; soy hydrolysate SE50MAF (DMV International), 1.5%; pH at 6.8 to 7.0.

Ten ml of seed medium in a 25×150 mm glass tube was inoculated with 0.2 mL of a frozen seed culture of BD240. The seed tube was incubated for 48 hours at 30° C., at 200 rpm using a gyro-rotary shaker with a 2-inch orbit.

A fermentation production medium of the following formulation was prepared by combining: dextrose (added after autoclaving), 1%; maltrin M180, 6%; soyflour, 1%; yeast extract, 0.6%; Gamaco (CaCO₃), 0.1%; L-proline, 0.4%; 3-(N-morpholino)propanesulfonic acid, 20.9 g/L; pH at 7.0.

Seed culture (0.5 mL) was inoculated into 25 ml of fermentation production medium in 250 ml Erlenmeyer flasks. These production flasks were incubated for 5 days at 26° C., 250 rpm using a gyro-rotary shaker (2-inch orbit).

B. Fermentor Fermentation

A seed medium of the following formulation was prepared by combining: dextrose (added after autoclaving), 1%; soluble starch, 2%; yeast extract (Difco), 0.3%; wheat hydrolysate (WGE80M, DMV International), 0.5%; soy hydrolysate SE50MAF (DMV International), 1.5%; pH at 6.8 to 7.0.

Frozen seed culture (0.5 mL) was inoculated into 250 mL of seed medium in a 2 liter Erlenmeyer flask. This seed flask was incubated for 48 hours at 30° C. at 200 rpm using a gyro-rotary shaker (2-inch orbit).

A fermentation production medium of the following formulation was prepared by combining: dextrose (added after autoclaving), 1%; maltrin M180, 6%; soyflour, 1%; yeast extract (Difco), 0.6%; Gamaco (CaCO₃), 0.1%; L-proline, 0.4%; Macol P2000, 0.1%; pH at 6.8 to 7.0.

The 250 mL of seed culture was inoculated into 8 liters of fermentation production medium in a 10 liter fermentor. The fermentation was incubated for 7 days at 26° C., agitation at 480-650 rpm, aeration at 1.0 volvol⁻¹min⁻¹(VVM).

### EXAMPLE 3

### Purification of Compound (I) from BD240

The cells from a 10 L culture of BD240 were pelleted by centrifugation. 5% Diaion-HP20 resin in water was added to the supernatant, and this was stirred at room temperature overnight. The HP20 resin was washed with methanol and the filtrate collected. To the cell pellet, 80:20 ethyl acetate methanol was added. This was repeatedly shaken and centrifuged, and the supernatant collected. The cell supernatant and the broth methanol filtrate were combined and concentrated in vacuo.

The crude extract was adsorbed onto silica (32-63μ, 60 Å), and fractionated by VLC. The compound was eluted with 5% methanol in dichloromethane. This material was concentrated in vacuo, adsorbed onto silica (32-63μ, 60 Å) and loaded onto a flash silica column (60 mm×250 mm). The compound was eluted with 2% methanol in dichloromethane. This material was concentrated in vacuo and loaded onto Sephadex LH-20 (60×400 mm, methanol). The column was initially washed with 300 ml methanol and 30 ml fractions were collected and monitored by LCMS. Early fractions which contained the compound of interest were collected and concentrated. This semi-pure material was chromatographed by preparative HPLC (YMC ODS-A 50×250 mm 10μ column, A: water B: methanol, gradient: 55% B to 70% B in 200 minutes, 30 ml/min). Compound I was identified through LCMS analysis of fractions. These fractions of interest were pooled, concentrated in vacuo to afford pure compound I (t_{R}=150 min, 220 mg).

Compound I can be purified from actinomycete strain LL-C31037 using similar methods.

### EXAMPLE 4

### Neuroprotective Properties of Compound (I) in Neuronal Cell Culture

Mesencephalic dopaminergic neuron cultures were prepared as previously described in Pong et al., J Neurochem. 69: 986-994 (1997). Embryonic day 15 (E15) rat fetuses were collected and dissected in ice-cold phosphate-buffered saline (PBS). The ventral piece of tissue compromising the mesencephalic dopaminergic region was dissected out. Dissected pieces of tissue were pooled together and transferred to an enzymatic dissociation medium containing 20 IU/ml papain in Earle's balanced salt solution (Worthington Biochemical, Freehold, N.J., U.S.A.) and incubated for 60 minutes at 37° C. After enzymatic dissociation, the papain solution was aspirated and the tissue mechanically triturated with a fire-polished glass Pasteur pipette in complete medium [equal volumes of minimum essential medium (MEM) and F-12 nutrient mixture (Gibco BRL) supplemented with 0.1 mg/ml apotransferrin and 2.5 μg/ml insulin] containing 2,000 IU/ml DNase and 10 mg/ml ovomucoid protease inhibitor.

High-Affinity Dopamine Uptake Assay

For dopamine uptake experiments, single-cell suspensions in complete media were seeded on poly-L-ornithine and laminin coated 24-well plates. The cultures were maintained for seven days prior to experimentation. Cultures were pretreated with various concentrations of the compound of formula (I) (washed with PBS and diluted with media to concentrations of 1 nM to 1000 nM) for 24 hours, then exposed to 10 μM the neurotoxin 1-methyl-4-phenylpyridinium (MPP⁺) for 1 hour to assess the neuroprotective effect of the compound of formula (I) in cell culture. Following the 1 hour incubation, media was exchanged three times and fresh compound was added for an additional 48 hours.

More particularly, after 48 hours growth of mesencephalic dopaminergic neuron cultures following MPP⁺ exposure, high-affinity ³H-dopamine uptake was performed using a modified method described by Prochiantz et al., Nature 293: 570-572 (1981). Cultures were washed with pre-warmed phosphate-buffered saline (PBS) containing 5.6 mM glucose and 1 mM ascorbic acid. Cultures were then incubated for 15 minutes at 37° C. with 50 nM ³H-dopamine (31 Ci/mmol, Du Pont-NEN, Wilmington, Del., U.S.A.). The cultures were washed twice with buffer and lysed with 0.5 N NaOH. The lysate was transferred to a scintillation vial containing Ultima Gold scintillation cocktail and radioactivity was determined with a liquid scintillation counter. Alternatively, culture lysates can be washed twice with buffer, incubated for 2 hours at room temperature with Optiphase Supermix scintillation cocktail (Wallac Scintillation Products, Gaithersburg, Md., USA), and radioactivity measured with a liquid scintillation counter.

TABLE 1
³H-DOPAMINE UPTAKE (% UNTREATED
CONTROL) IN CULTURED DOPAMINERGIC
NEURONS AFTER MPP+ INDUCED TOXICITY

| | ³H-DOPAMINE UPTAKE |
|---|---|
| TREATMENT | (% UNTREATED CONTROL) |
| Untreated control | 100% |
| 10 uM MPP⁺ | 40% |
| 1 nM meridamycin | 47% |
| 10 nM meridamycin | 51% |
| 100 nM meridamycin | 51% |
| 1000 Nm meridamycin | 64% |

MPP⁺-induced neurotoxicity in a ³H-dopamine uptake cell assay was measured. Neuroprotective effects in cell culture were measured by addition of the compound (1 nM to 1000 nM) to the culture in the presence of MPP. As shown in Table 1, the compound was neuroprotective against MPP⁺-induced neurotoxicity in cultured dopaminergic neurons, with an EC₅₀ of 555 nM relative to a maximum protection (84% uptake) afforded by 10 ng/mL GDNF.

### EXAMPLE 5

### Neuroregenerative Properties of Compound (I) in Neuronal Cell Culture

Dissociated cortical neuron cultures were prepared as previously described [Pong et al., Exp Neurol. 2001 September; 171(1):84-97 (2001)]. Briefly, embryonic day 15 rat fetuses were collected and dissected in ice-cold PBS. Dissected cortices were pooled together and transferred to an enzymatic dissociation medium containing papain. After 30 min, the tissue was mechanically triturated with a fire-polished glass Pasteur pipette. Single-cell suspensions in complete media were seeded on poly-L-ornithine and laminin coated 96-well plates. 24 hours later, cultures were treated with various concentrations of compound of formula (I) for 72 hours. The cultures were then fixed and stained with a neurofilament primary antibody and a peroxidase-tagged secondary antibody. A peroxidase substrate (K-Blue Max) was added and the colorimetric change was measure on a colorimetric plate reader.

Dissociated cortical neuron cultures were prepared as previously described (Pong et al., 2001). Briefly, embryonic day 15 rat fetuses were collected and dissected in ice-cold PBS. Dissected cortices were pooled together and transferred to an enzymatic dissociation medium containing papain. After 30 min, the tissue was mechanically triturated with a fire-polished glass Pasteur pipette. Single-cell suspensions in complete media were seeded on ploy-L-ornithine and laminin coated 96-well plates. 24 hours later, cultures were treated with various concentrations of compound for 72 hours. The cultures were then fixed and stained with a neurofilament primary antibody and a peroxidase-tagged secondary antibody. A peroxidase substrate (K-Blue Max) was added and the calorimetric change was measure on a calorimetric plate reader.

TABLE 2
NEUROFILAMENT CONTENT (FOLD-INCREASE ABOVE
UNTREATED CONTROL) IN CULTURED CORTICAL NEURONS

| | NEUROFILAMENT CONTENT |
|---|---|
| | (FOLD-INCREASE ABOVE |
| TREATMENT | UNTREATED CONTROL) |
| Untreated control | 1.0 |
| 10 nM meridamycin | 1.71 |
| 100 nM meridamycin | 2.24 |
| 1 μM meridamycin | 2.32 |
| 10 μM meridamycin | 2.56 |

As shown in Table 2, addition of the compound to neuronal cells increased neuronal survival in cultured cortical neurons, with an EC₅₀ of 12 nM.

### EXAMPLE 6

### Neuroregenerative Properties of Compound (I) in Cultured Cortical Neurons

Dissociated cortical neuron cultures were prepared as previously described (Pong et al., cited above, 2001). Briefly, embryonic day 15 rat fetuses were collected and dissected in ice-cold PBS. Dissected cortices were pooled together and transferred to an enzymatic dissociation medium containing papain. After 30 minutes, the tissue was mechanically triturated with a fire-polished glass Pasteur pipette. Single-cell suspensions in complete media were seeded on poly-L-ornithine and laminin coated 96-well plates. After 24 hours, cultures were treated with various concentrations of the compound of formula I for 72 hours. The cultures were then fixed and stained with an anti-tubulin primary antibody (TUJ-1) and a fluorescent-tagged secondary antibody. Neurite outgrowth was determined by using the Enhanced Neurite Outgrowth (ENO) algorithm with the Cellomics ArrayScan and expressed as total neurite length per cell.

TABLE 3
Total Neurite Length (% Above Control) in Cultured Cortical Neurons

| Treatment | Total Neurite Length (% Above Control) |
|---|---|
| 10 nM Compound | 19% |
| 100 nM Compound | 40% |
| 1 μM Compound | 113% |
| 10 μM Compound | 152% |

### EXAMPLE 7

### Neuroregenerative Properties of Compound (I) in Cultured Dorsal Root Ganglia

Dissociated dorsal root ganglia cultures were prepared as previously described [A. Wood et al., “Stimulation of neurite outgrowth by immunophilin ligands: quantitative analysis by Cellomics Array scan” Society for Neuroscience (2004), abstract 104.3] Briefly, postnatal day 3-5 rat pups were euthanized. The spinal columns were removed and individual dorsal root ganglia (DRG) were dissected out. Dissected DRG were pooled together and transferred to an enzymatic dissociation medium containing papain. After 60 minutes, the tissue was mechanically triturated with a fire-polished glass Pasteur pipette. Single-cell suspensions in complete media were seeded on poly-L-ornithine and laminin coated 96-well plates. After 24 hours, cultures were treated with various concentrations of the compound of formula I for 72 hours. The cultures were then fixed and stained with an anti-tubulin primary antibody (TUJ-1) and a fluorescent-tagged secondary antibody. Neurite outgrowth was determined by using the Enhanced Neurite Outgrowth (ENO) algorithm with the Cellomics ArrayScan and expressed as total neurite length per cell.

TABLE 4
Total Neurite Length (% Above Control) in
Cultured Dorsal Root Ganglia

| Treatment | Total Neurite Length (% Above Control) |
|---|---|
| 10 nM Compound | 4% |
| 100 nM Compound | 9% |
| 1 μM Compound | 14% |
| 10 μM Compound | 24% |

When ranges are used herein for physical properties, such as molecular weight, or chemical properties, such as chemical formulae, all combinations and subcombinations of ranges and specific embodiments therein are intended to be included. All publications cited in this specification, and the deposits, are incorporated herein by reference. While the invention has been described with reference to particular embodiments, it will be appreciated that modifications can be made without departing from the spirit of the invention. Such modifications are intended to fall within the scope of the appended claims.

1. A method for treatment of a neurological disorder comprising administering to a mammal an effective amount of a meridamycin or a salt thereof.

2. The method according to claim 1, wherein the meridamycin has the structure of formula (I):

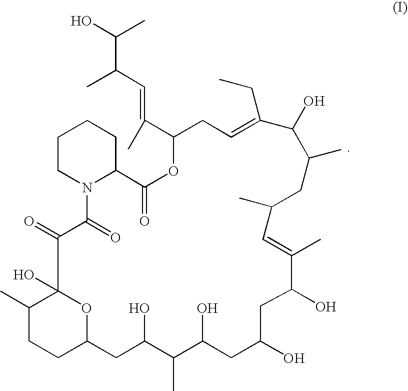

3. The method according to claim 1, wherein the meridiamycin has one or more of the following characteristics:
an apparent Molecular Formula: C₄₅H₇₅O₁₂N
Molecular Weight: Positive Ion Electrospray m/z=844.8 (M+Na)⁺; Negative Ion Electrospray MS m/z=821.1 (M−H)⁻; High Resolution Fourier Transform MS m/z=822.53637 (M+H)⁺
Ultraviolet Absorption Spectrum: λₘₐₓ nm (acetonitrile/water)=210 nm, end absorption;
Optical Rotation [α]²⁵_{D}−1.4 (c 1.0, MeOH); and
a proton magnetic resonance spectrum of FIG. 1.

4. The method according to claim 1 further comprising identifying a mammal suffering from the neurological disorder.

5. The method according to claim 1 further comprising assessing a degree of neurodegeneration in a mammal.

6. The method according to claim 5 wherein the assessment is made before administering said compound.

7. The method according to claim 5 wherein the assessment is made after administering said compound.

8. The method according to claim 1 wherein the neurological disorder is epilepsy, stroke, cerebral ischemia, cerebral palsy, Alper's disease, Parkinson's disease, Alzheimer's disease, Huntington's disease, amyotrophic lateral sclerosis, multiple sclerosis, dementia with Lewy bodies, Rhett syndrome, neuropathic pain, spinal cord trauma, or traumatic brain injury.

9. The method according to claim 1 wherein the neurological disorder is senile dementia, dementia with Lewy bodies, mild cognitive impairment, Alzheimer's disease, or cognitive decline.

10. The method according to claim 1 wherein the compound is admixed with one or more pharmaceutically acceptable carriers, excipients, or diluents.
